(19) United States
(12) Patent Application Publication
(10) Pub. No.: US 2025/0359751 A1
(43) Pub. Date: Nov. 27, 2025

(54) Automated Adaptive Method To Standardize Visual Acuity Testing And Reporting

(71) Applicants: James W. O'NEIL, Phoenix, AZ (US); Richard S. TIRENDI, Paradise Valley, AZ (US)

(72) Inventors: James W. O'NEIL, Phoenix, AZ (US); Richard S. TIRENDI, Paradise Valley, AZ (US)

(21) Appl. No.: 19/231,269

(22) Filed: Jun. 6, 2025

Related U.S. Application Data
(63) Continuation-in-part of application No. 17/240,382, filed on Apr. 26, 2021, now abandoned.

(51) Int. Cl. A61B 3/032

(52) U.S. Cl. CPC A61B 3/032

(57) ABSTRACT

A system and method for evaluating visual acuity using a computerized or otherwise digitalized adaptive vision test is disclosed. The digitalized system presents users with a series of displays wherein each display presents three vertically aligned optotypes wherein the top optotype is the largest, the middle optotype is smaller, and the bottom optotype is of size between the top and middle optotypes. Alternatively, at times all three optotypes are equal in size. The system may adjust the difficulty of the upcoming display(s) based on user responses, such that correct identification of optotypes may trigger a reduction in the middle letter's size, accelerating convergence to the user's acuity threshold while minimizing visual fatigue.

### CLAIM OF PRIORITY

The present continuation-in-part application includes subject matter disclosed in and claims priority to U.S. patent application Ser. No. 17/240,382 filed Apr. 26, 2021, entitled “Automated Adaptive Method To Standardize Visual Acuity Testing And Reporting”, which claims priority to U.S. provisional patent application Ser. No. 63/020,013, filed May 5, 2020, entitled “An Automated Adaptive Algorithm Method To Standardize Visual Acuity Testing And Reporting”, both incorporated herein by reference, and which describe inventions made by the present inventors.

### BACKGROUND OF THE INVENTION

### 1. Field of the Invention

The present invention relates to visual acuity measuring systems and methods, and more particularly to a computer-assisted adaptive system and method for measuring visual acuity with progressive screens displays.

### 2. Description of Related Prior Art

Assessment of visual acuity is a critical component of an eye examination or vision screening. Visual acuity tests presently known in the art typically include a subjective assessment using standard charts, such as the Snellen or LogMAR charts.

The Snellen eye chart is a standardized tool consisting of rows of optotypes arranged vertically, with each row displaying progressively smaller optotypes. The Snellen chart is designed to correspond to specific visual acuity levels, such that each descending vertical row corresponds to a progressively lower visual acuity level. The first level may correspond to a visual acuity score of 20/200 (which in some countries may correspond with legal blindness, being able to see from twenty feet what someone with average vision can see from two hundred feet) while the bottom level corresponds to a visual acuity level of 20/10 (exceptional acuity, being able to see from twenty feet what someone with average vision can see with from ten feet), with the intervening rows corresponding to visual acuity levels between 20/200 or 20/400 and 20/10, respectively descending from 20/100 to 20/16.

While each line of the Snellen chart is calibrated to ensure standardized testing, the Snellen chart is a suboptimal tool for measuring visual acuity. There are numerous disadvantages of Snellen charts, partially due to inconsistent letter size progression and an uneven number of optotypes per line. Poor vision lines, such as 20/200 and 20/400, typically contain only one or two letters, whereas acuity lines with smaller optotypes may contain up to eight letters. Lines with multiple letters may lead to inaccurate testing, as the additional optotypes can provide visual context cues that make them easier to read, potentially resulting in an overly optimistic assessment of visual acuity.

Additionally, the Snellen test is conducted using a line assignment method, rather than a letter-by-letter scoring method, such that the visual acuity score is based on the “smallest” line of optotypes one can read correctly. Optotypes read correctly on smaller lines (below the last full line read) are not counted in the score. Therefore, using the line assignment method with variable letters per line, a change in acuity of one letter can result in a change of vision of an entire line. The line assignment method also prevents measurement of visual acuity on a fine scale. Due to the lack of standardized progression between lines, Snellen visual acuity is challenging to assess statistically. Parametric analysis cannot be performed with this decimal progression sequence, even if converted to another form.

Furthermore, the Snellen test is inconsistent in letter size progression, as the size of the letters does not change in a perfectly linear or logarithmic fashion. The irregular and arbitrary progression of letter sizes between lines introduces considerable error when changing the viewing distance of the chart, leading to an overestimation of vision at the lower end of acuities. Moreover, a loss or gain in a line of vision does not have the same meaning in different parts of the chart.

The Snellen test is also subpar because the optotypes on a Snellen chart are not always of the same legibility. Some letters, such as C, D, E, G, O, are easier to read than others, such as A, J, and L, leading to inconsistent results.

In optotype-based visual acuity tests, when optotypes are spaced too closely, optotypes may interfere with one another, affecting visual clarity. This effect, known as the crowding phenomenon, occurs when adjacent contours impair one's ability to identify individual optotypes, thereby diminishing acuity. The extent of crowding varies across the Snellen chart. For example, lines representing poorer visual acuity typically have fewer and more widely spaced optotypes, resulting in minimal crowding. In contrast, lines representing better acuity often contain more optotypes positioned closely together, increasing the crowding effect. In an ideal visual acuity test, each line should pose an equivalent challenge to the patient, aside from optotype size. However, inconsistencies in the number of letters and their spacing compromise this uniformity. As a result, patients with impaired central vision, who may accurately identify isolated letters, may struggle to read complete lines, not due to an inability to see the optotypes, but because of the disruptive influence of crowding.

Other issues with the “Snellen Chart” are due to the fact that the “Snellen chart” is not standardized. Therefore, charts from different manufacturers may use different fonts, different letters, and different spacing ratios. Additionally, different charts may be illuminated or projected differently.

The LogMAR chart is an alternative to the Snellen chart. Unlike the Snellen chart, the LogMAR chart provides a test with a logarithmic progression of optotype size. The name of the chart is an abbreviation for “logarithm of the Minimum Angle of Resolution”. The chart is designed to enable a more accurate estimate of acuity than the Snellen chart). When using a LogMAR chart, visual acuity is scored with reference to the logarithm of the minimum angle of resolution. For example, a user who can resolve details as small as 1 minute of visual angle scores LogMAR 0, since the base-10 logarithm of 1 is 0; while an observer who can resolve details as small as 2 minutes of visual angle (i.e., reduced acuity) scores LogMAR 0.3, since the base-10 logarithm of 2 is near-approximately 0.3.

Unlike the Snellen chart, the LogMAR chart has a logarithmic progression of optotypes, standardized and equal spacing between letters and lines, exactly five letters per line, an optotype-by-optotype scoring method, and uniform crowding across all lines. Therefore, a LogMAR chart-based visual acuity test offers precise measurements, detects small changes in vision, and reduces bias caused by letter arrangement, crowding, or inconsistent line difficulty.

For pre-literate children, non-literate or non-verbal individuals, and those unfamiliar with the alphabet being used on a standard eye chart, a tumbling E chart may be used for a visual acuity screening. A tumbling E chart is an optotype chart that exclusively relies on the letter “E”, presented in various rotations, to test a user's visual acuity. The “E” may be rotated such that each optotype is pointing up, down, left, or right. Traditional tumbling E charts are similar to Snellen charts and do not rely on LogMAR principles. But tumbling E charts, commonly referred to as “ETDRS-style Tumbling E charts” are designed following the LogMAR principles as described above. Such “ETDRS-style Tumbling E charts” rely on a logarithmic size progression for each line set, have five optotypes per line, include equal spacing between letters and between lines, and are scored letter by letter rather than line by line.

Neither traditional and ETDRS-style Tumbling “E” charts are recommended by the American Academy of Ophthalmology (AAO), the American Academy of Pediatrics (AAP), the American Association for Pediatric Ophthalmology and Strabismus (AAPOS), or the Research to Prevent Blindness charity. Tumbling E charts are generally discouraged due to accuracy concerns. When users guess the direction of the “E,” there is a twenty-five percent chance that a user may guess the direction correctly, as the optotype can only point in one of four directions. Moreover, because the letter's orientation is limited to either horizontal or vertical, partial recognition may occur near the threshold of visual acuity. In these cases, users may discern the general orientation of the “E's” arms without clearly identifying the direction of the opening, effectively increasing the chance of a correct guess to nearly 50%. Furthermore, young children, the primary target users of tumbling “E” charts, often exhibit right/left confusion, resulting in errors when matching or pointing out the direction of the tumbling E.

To overcome the confusion associated with the right/left orientation of Tumbling “E” charts, an alternative visual acuity test, designed for young children or those unfamiliar with the Roman alphabet, may be used. This alternative test is the “HOTV chart.” HOTV charts are visual acuity tests that rely on only four capital letters: “H”, “O”, “T”, and “V”. The four capital letters are chosen as they are symmetrical and easily distinguishable, thereby minimizing confusion. To perform the test, users may either name the letter (if they can) or use the matching method, whereby they match the letter being displayed to a card with the same letter. HOTV charts are available in LogMAR format for research or standardized clinical use.

While in clinical setting, “HOTV” charts are preferred over Tumbling “E” charts, “HOTV” charts are not typically recommended for children over age 7 with age-appropriate development, or for literate adults, as there is a recognized 0.06 LogMAR (3 letters) overestimation of visual acuity using HOTV symbols as compared to ETDRS letters.

Another alternative to Tumbling “E” charts, particularly for children and other illiterate individuals, is the Glasgow acuity chart. The Glasgow acuity chart includes six letters, rather than the four included in the “HOTV” chart, such that a selection of 6 possible letters reduces the risk of random guessing for young children. Like HOTV, the letters used in Glasgow (X, V, O, H, U, and Y) were chosen to be symmetrical around the vertical midline to avoid right-left confusion in younger children. Still, children may misname and confuse letters; therefore, some prefer using picture charts, such as the LEA picture chart.

A detailed discussion of the use of computer-assistive methods are discussed at length in The Ohio State University Thesis in the Graduate Program in Vision Science by Erin Andrews, 2017, entitled Computer-assisted Adaptive Methods of Measuring Visual Acuity (herein incorporated by reference).

Given the variety of visual acuity charts and protocols, results from the various testing methods may yield varying scores for the same user, even in the absence of actual visual changes. Therefore, there is a significant need for testing modifications that minimize test-to-test variability to improve testing accuracy. One approach for improved testing may involve adaptive testing, such that testing procedures are adjusted based on the user's response to a previous question. Some adaptive testing protocols involve testing near the estimated threshold, adjusting the optotype around the estimated threshold until an exact visual acuity score is determined.

Such adaptive protocols are often complex and require formalized and extensive training to administer.

To overcome the complexities associated with adaptive protocols, testing may be digitalized such that optotype adjustments are computer-generated and adjusted by a computer-based algorithm. Such computer-generated tests may improve the efficiency and repeatability of visual acuity testing and do not require extensive training to administer. However, computer-generated adaptive visual acuity tests, currently known in the art, have not gained widespread acceptance as a mainstream approach to visual acuity testing for various reasons. Computer-generated adaptive visual acuity tests presently known in the art are subpar due to the fact that the currently available tests require a significant amount of time to administer. Providers often lack the time necessary to administer such tests, which can require approximately ten minutes of testing per eye. Furthermore, because digital adaptive visual acuity tests require lengthy administration, patient fatigue may develop, potentially leading to inaccurate results that suggest a patient's vision is worse than it truly is. Additionally, although digital adaptive visual acuity tests do not require expert-guided administration, many protocols currently known in the art are only available for use through a professional office, and on a computer, rather than on a smartphone or tablet for widespread at-home testing.

Those digitized visual acuity tests that are available on a smartphone or tablet, such as the “PEEK app” and the “Vision@Home,” do not follow either LogMAR or ETDRS design principles, and therefore do not meet the necessary accuracy and reliability standards for proper visual acuity testing. Such smartphone and tablet-based apps are often inaccurate and do not meet the standards for use in telehealth.

There is a significant need for a digital adaptive visual acuity testing platform equipped with an algorithm optimized for mobile devices. This algorithm and testing framework must meet or exceed the accuracy benchmarks established by the ETDRS and LogMAR protocols. Such a platform is essential for enabling reliable and precise visual acuity assessments across diverse environments, including settings outside professional eye care facilities. Such a platform may enable non-specialist users, such as parents and other caregivers lacking formal training, to effectively conduct visual acuity testing e, ensuring consistent and accurate results regardless of the examiner's expertise.

### SUMMARY OF THE INVENTION

The herein described platform is preferably a digital platform for determining a subject's visual acuity. The platform may include a screen that presents vertical groups of a series of grouped and regrouped optotypes to a user. The screen may be programmed with a plurality of algorithms, such that the plurality of algorithms is programmed to analyze a user's responses to the presented groups and regroups of optotypes to determine future optotype presentations for testing. The plurality of algorithms may include a first algorithm for an adaptive threshold determining phase, herein referred to as a first descending phase of testing, and a second algorithm for a second threshold refining phase, herein referred to as the breakout phase of testing. The plurality of algorithms may also include an algorithm for calculating a user's visual performance.

The digital platform may be implemented on a personal device or on a professional device. In some embodiments, the digital platform may be implemented on a personal smart device, a smartphone, a tablet, a computer, virtual reality goggles, red/blue lenses, polarized glasses, and/or a screen. The digital platform may be programmed to test vision in a user's right and left eye, or alternatively the platform may be programmed to test vision in a single eye.

The digital platform may present optotypes in a vertical column, such that one optotype is set per line of the column. The vertical column may be comprised of three optotypes set in a single vertical row. In some embodiments, the vertical column may be comprised of between one and three optotypes, set in a single vertical row. The platform may display a sequence of screens, such that each screen presents a vertical column of optotypes, such that the size of the optotypes varies from screen to screen. In some embodiments, each optotype on a single screen is a different size. In some embodiments, the optotypes on a single screen may be equal in size to one another.

In preferred embodiments, the size of the optotypes adapts between screens, in response to the correctness of a user's response to presented optotypes. In some embodiments, the first algorithm determines the appropriate optotype size for presentation based on a user's response to presented optotypes. In some embodiments, the second algorithm may calculate the optotype size for presentation during the second refining threshold phase, based on the user's responses to the optotypes presented during the first threshold-determining phase. In preferred embodiments, the digital platform may be programmed to present optotypes in accordance with a LogMAR protocol for decreasing and increasing optotype size. In some embodiments, the difference in size between optotypes is approximately 0.1 LogMAR.

In some embodiments, particularly for measuring myopia progression, the presented optotypes may vary in color. In some embodiments, the optotypes on a single display vary in color. In some embodiments, the colored optotypes may be displayed with a black, or otherwise colored background.

In some embodiments, the optotypes may be positioned in a vertical column, such that the largest optotype is positioned at the top of the column, the mid-sized optotype is positioned at the center of the column, and the smallest optotype is positioned at the bottom of the column. In some embodiments, the digital platform may assess a user's visual performance by presenting a series of vertically arranged columns of optotypes to a user, in two stages: a first threshold determining stage, and a second threshold refining stage. In such embodiments, each column may include optotypes of varying sizes, or alternatively optotypes of the same size. In some embodiments, optotypes within a single column may vary in size during the first threshold determining stage and may be uniform in size during the second threshold refining stage. In preferred embodiments, during both stages, the selection of each subsequent column may be dynamically determined based on the user's response to the preceding column. It is preferable that each display module includes a single vertical column, such that the display platform displays optotypes in a single vertical row, with each line of the column including only a single optotype.

The user's visual performance may be calculated based on a user's responses to the displayed optotypes. Additionally, the time it takes a user to respond to a displayed optotype may also impact the user's visual performance score.

The method for determining a user's visual performance score may include showing a user a digital platform such that the platform displays a series of screens, wherein each screen presents a vertical column composed of optotypes, such that on each subsequent screen optotypes are grouped and regrouped in vertical columns. It is preferable that the platform first presents a series of screens with shifting optotype sizes to determine a user's general vision threshold, after which the platform may present a series of screens with a narrower size range of optotypes to further refine the user's visual performance score.

In some embodiments, the presentation and scoring may be completed in a total timeframe of between two and twelve minutes for testing a user's right and left eye. In some embodiments, the method may involve the user covering one eye before viewing the series of displays, such that the vision in each eye is tested separately. The method may test a user's visual acuity, myopia progression, near vision, contrast sensitivity, vernier acuity, stereopsis, convergence, accommodative amplitude, color, focal length determination, and/or binocularity.

In some embodiments, in the first series of screens, the top, middle, and bottom optotypes represent three adjacent LogMAR vision lines. Repeated testing via grouping and regrouping a subsequent set of three vertically oriented optotypes on a display may be administered in the first series of screens. The smallest of the optotypes tested may be smaller than an optotype that was failed on a previous display.

In some embodiments, a LogMAR threshold of visual acuity may be determined, and an optotype more than 0.1 LogMAR removed from the LogMAR threshold of visual acuity may be displayed, such that users are tested with optotypes lower than their determined or estimated threshold of visual acuity or other vision scores. In some embodiments, the steps of grouping and regrouping in the first series of screens may be repeated until at least ten optotypes are displayed, such that five optotypes are evaluated on the LogMAR threshold with at least three out of five correct, and whereby at least five optotypes on the adjacent line immediately below the LogMAR threshold are failed with less than three out of five correct.

In some embodiments, the second series of screens may involve further displaying three optotypes above the LogMAR threshold determined by the first series of screens, in descending fashion after the LogMAR threshold is obtained. In some embodiments, incongruent data points may be identified, such as either an optotype larger than the LogMAR threshold that failed, or an optotype smaller than the LogMAR threshold that passed. Incongruent data points may be weighed by being assigned values as false positives or false negatives depending on the incongruent point's distance from the LogMAR threshold.

In some embodiments, the test may be administered by repeatedly displaying three vertically arranged optotypes on a screen. The largest of the three optotypes may be set at the top of the column and the smallest optotype may be set at the bottom of the column.

In some embodiments, the test may be administered via an electronic device that includes a three-letter display with one letter each from three adjacent LogMAR vision lines displayed. In preferred embodiments, the top optotype is larger than a middle optotype, and the middle optotype is larger than the bottom optotype. In some embodiments, the top letter may be 0.02 LogMAR larger than the middle letter, and the middle letter may be 0.02 LogMAR larger than the bottom letter.

### BRIEF DESCRIPTION OF THE DRAWINGS

The present invention will be described with greater specificity and clarity with reference to the following drawings, in which:

FIG. 1 illustrates a screen presenting optotypes in accordance with an embodiment of the present invention.

### DETAILED DESCRIPTION OF THE PREFERRED EMBODIMENTS

The present disclosure describes an adaptive visual acuity testing platform, algorithm, and protocol, herein referred to as “the platform”, suitable for use on mobile devices, including but not limited to smartphones and tablets, such that the herein described platform may facilitate vision testing in a variety of testing locations, by individuals not skilled in the art of visual acuity testing. The described adaptive platform produces results that are at least as accurate as those obtained through traditional ETDRS and LogMAR protocols, while also being approximately forty percent faster to administer, regardless of examiner training or experience. Embodiments such as those described herein have been tested, with the results discussed in Analysis of the Reliability and Repeatability of Distance Visual Acuity Measurement with EyeSpy 20/20 by Balamuri Vasudevan, et al., Clinical Ophthalmology 2022:16, 1099-1108 (herein incorporated by reference), and Inter-Rater Reliability of EyeSpy Mobile for Pediatric Visual Acuity Assessments by Parent Volunteers by Elyssa Rosenthal, et al., Clinical Ophthalmology 2024:18, 235-245 (herein incorporated by reference).

The herein described system offers significant advancement over traditional and existing computerized visual testing methodologies. Unlike conventional sequential display methods, which primarily present a single optotype for user assessment, the described protocol presents groupings of optotypes to determine and narrow down appropriate parameters for optotypes to be presented in subsequent test presentations.

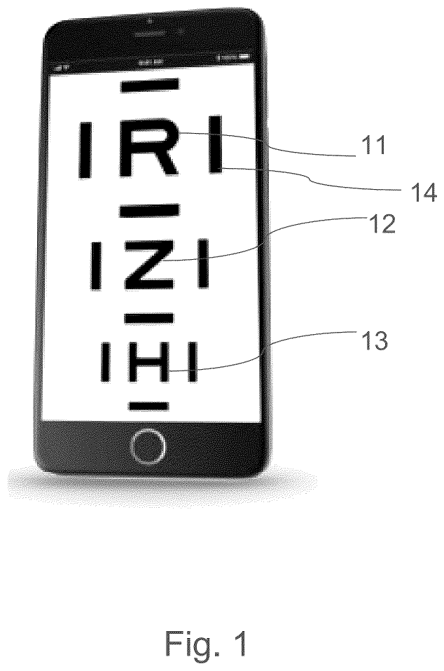

In some embodiments, the herein-described testing system is designed to display, preferably, three optotypes simultaneously, with each optotype being different in size. As seen in FIG. 1, in some embodiments, the three differently sized optotypes may be arranged in a vertical, column-like orientation. In some embodiments, the largest optotype is presented on the top of the column, the smallest optotype is presented in the middle of the column, and an intermediate optotype, sized between the largest and smallest of the three, is presented on the bottom of the column. In some embodiments, as seen in FIG. 1, largest optotype 11 is presented on the top of the column, intermediate sized optotype 12 is presented in the center of the column, and smallest optotype 13 is presented at the bottom of the column. In preferred embodiments, the difference in the size of the three presented optotypes is minimal, preferably having a difference in size of approximately 0.1 LogMar. Such a vertical arrangement is preferable, as a vertical arrangement leverages the user's natural tendency to read from top to bottom, thereby aligning with the innate characteristics of visual processing. Additionally, the vertical display arrangement may be formatted to be compatible with the screen size constraints of smartphones.

Other app-based visual acuity tests currently known in the art are subpar due to scoring inaccuracies, as they do not meet the standards of the EDTRS tests combined with the LogMar scoring method used in clinical settings. In preferred embodiments, the herein-described platforms may match the standards set by both EDTRS and LogMAR, in part by employing the same optotypes relied upon by the EDTRS system. The EDTRS system and the described platform both preferably utilize a specific set of ten optotypes that are specially balanced for their difficulty and legibility properties. Namely, the EDTRS test, and the platform described herein only uses the optotypes C, D, H, K, N, O, R, S, V, and Z. The aforementioned optotypes are preferable, as they have approximately equal recognizability when presented at the same size, such that no one optotype is inherently “easier” or “harder” to recognize than another, reducing bias in test results. I

Additionally, the optotypes used in EDTRS testing, as well as the platform described herein, preferably encompass a range of visual features, including straight lines (H, K), curves (C, O), diagonals (Z, V), and combinations thereof, as discussed above. Such variation mimics real-world visual complexity, helping assess true visual capability, rather than just specific pattern recognition, as is the case with visual acuity app-based tests currently known in the art.

The specific optotypes chosen for ETDRS testing, and preferably for the herein described platform, are also specifically selected not to include letters with similar shapes such as both O and Q, or mirrored letters such as both “b” and “d”, to prevent letter confusion, guessing, left-right confusion, and partial recognition.

In some embodiments, the platform described herein may be programmed to enable users to select from a plurality of optotype and/or eye test options, such that users may select the eye exam most suited to their needs. For example, in some embodiments, users may choose from ETDRS style optotypes, tumbling “E” style optotypes, HOTV optotypes, symbols etc.

The herein described platform also meets the reliability standards provided by the LogMAR scoring system, particularly by relying on “letter by letter” scoring, rather than calculating a user's score based on the correctness of an entire line, or a whole set of optotypes. Additionally, the herein described platform presents letters differing in size by approximately 0.1 logMar, allowing for increased sensitivity, similar to traditional LogMAR testing.

While, as described above, the ETDRS test combined with the LogMar scoring method is the quintessential model for precision in visual acuity testing, such tests are time-consuming and tedious to perform. The ETDRS test employs a two-phase process consisting of screening and testing. The test assesses visual acuity by identifying the smallest line on which all five letters are correctly recognized and the largest line on which all five letters are missed. Intermediate lines are also evaluated to derive a precise visual acuity score, wherein up to seventy optotypes are presented for testing.

ETDRS tests are not only tedious to administer, but ETDRS-style testing may also cause fatigue in users, as identifying approximately seventy optotypes can be wearisome for young children and those with short attention spans. User fatigue may compromise test reliability, as users may be answering incorrectly due to fatigue, boredom, and distractions, rather than poor visual acuity.

“The platform”, described herein, preferably integrates the scoring accuracy of the ETDRS-style (Early Treatment Diabetic Retinopathy Study) testing method and the LogMAR (Logarithm of the Minimum Angle of Resolution) scoring system with the expediency of an app-based testing format. This combination ensures precise visual acuity measurement while offering the benefits of a streamlined, expedient, digital interface that may be used at home, in school settings, and administered by non-skilled users.

To initiate the platform, it is preferable that users first calibrate the platform to suit their needs, including but not limited to calibrating the starting “line” for testing, the appropriate distance, etc. After proper calibration and initiation, a user may cup one hand over their eye, or alternatively cover one eye in another appropriate manner, after which testing may be initiated.

In preferred embodiments, the platform may first initiate a threshold determining phase, also known as a “descending phase”, or “descending staircase phase” wherein an adaptive series of vertical columns of optotypes may be presented to a user to determine an approximate visual acuity threshold, after which a second threshold refining phase may be initiated by the platform, such that the initial determined threshold may be further optimized. The aforementioned threshold refining phase may also be referred to herein as the “breakout phase”. The rules for determining optotype size progression are herein described below.

To maintain accuracy and efficiency, the platform begins with the “descending phase” of testing, wherein the logic flow, which determines the appropriate optotype size to present during testing, is established. This descending phase is preferable for optimizing the testing sequence for the subsequent “testing phase”, also known as the “breakout phase”.

To administer the descending phase, optotypes corresponding to a specific visual acuity level are presented. Unlike the testing phase, described below, scoring during the descending phase serves to determine the appropriate starting point for testing, rather than final visual acuity.

To expedite testing and prevent user fatigue, in some embodiments, the descending phase may begin at a mid-level visual acuity score, such as 20/63. Users may choose to begin the descending phase with optotypes corresponding to any visual acuity line, as long as the visual acuity selected line is presentable given the user's screen size and distance capabilities. In some embodiments, when displaying larger optotypes, some screens may only display one or two optotypes, rather than the preferred three. Starting at the mid-level acuity line enables the test to efficiently assess the user's capabilities without presenting unnecessarily large optotypes. If the user cannot correctly identify optotypes at this mid-level, larger optotypes may then be displayed. Conversely, if the user successfully identifies the mid-level optotypes, the descending phase progresses with the presentation of consecutively smaller optotypes. Such an adaptive approach maximizes testing efficiency and expedites the assessment process by adjusting the starting point of testing based on the user's responses.

During the aforementioned descending phase, optotypes are presented in a vertical array, with preferably two or three consecutive optotypes stacked on top of one another, as shown in FIG. 1. Embodiments with less than two optotypes and more than three optotypes may be appropriate for particular screen sizes and are included in the scope of the present invention. The largest optotypes are positioned at the top, and the progressively smaller ones are placed below. As the user identifies the array of three optotypes, the screen changes to display a new screen with progressively smaller optotypes. In preferred embodiments, the same-sized optotypes will be presented twice, on different screens, ensuring that a user's response to a particular optotype is accurate and not related to guessing, letter confusion, or partial recognition. The descending phase, also referred to as the “descending staircase phase,” continues until descending phase presentation endpoints are met, wherein users fail to identify one or more of the displayed optotypes.

In preferred embodiments, to conclude the descending phase, such that the platform may continue with the visual acuity “test” phase, also known as “the breakout phase”, the platform must display at least two letter presentations at three consecutive visual acuity levels, such that two equally sized optotypes are presented at each of three descending visual acuity levels. In some embodiments, depending on a user's visual acuity, the initial descending phase, which sequentially presents progressively smaller optotypes, may need to be repeated more than once, particularly if the user's responses are inconsistent.

In some embodiments, if a user is determined to be at a particular visual acuity “level” during the descending phase, the platform may retain all the data from the presentations for later visual acuity score calculation. However, the scoring method used during the descending phase is intended to determine the optimal optotypes for presentation during the testing/breakout phase and differs from the scoring method used during the testing phase to determine a user's visual acuity.

Before a user progresses from the descending phase to the breakout phase, a set of criteria must be met. In some embodiments, the platform requires a minimum of two-letter presentations at three consecutive optotype levels to be displayed during the descending “staircase” phase, as described above. If the minimum two-letter presentation requirement is not met, the “staircase” may need to be repeated more than once to gather sufficient data. Once the aforementioned minimum criteria are met, the system evaluates prioritized rules to determine if the breakout stage is warranted, such that once a user satisfies one of the prioritized rules, the user may progress to the “breakout phase” described below. In a preferred embodiment, the system may evaluate six prioritized rules sequentially. In other embodiments, there may be more or fewer than six rules. In preferred embodiments, the rules may be evaluated sequentially, in order of priority, such that rule one is evaluated first, rule two is evaluated second, and so forth.

In preferred embodiments, rule one requires that there be at least two correct responses among the six presentations for the three displayed optotype sizes. If rule one is met, the platform may be programmed to advance the user to the refining “breakout phase” as described below. If the rule is not met, the platform may evaluate whether the criteria for rule two are met.

In preferred embodiments, rule two may examine whether there are at least two correct responses for the two smaller currently displayed sizes, and for the line one size smaller than the currently displayed smallest size. If rule two is met, the platform may be programmed to advance the user to the “breakout phase” as described below. If rule two is not met, the platform may evaluate whether the criteria for rule three are met.

Rule three requires that there be at least six responses total, the six responses being at least two responses for the smallest size “currently” being displayed, additionally there must be at least two responses for the “line” one size smaller than the smallest optotypes currently being shown, and at least two responses for the “line” two sizes smaller than the smallest optotype “currently” being displayed. Among the six said responses, two of the responses must have been correct. If rule three is met, the platform may be programmed to advance the user to the “breakout phase” as described below. If rule three is not met, the platform may evaluate whether the criteria for rule four are met.

Rule four is met when the two largest-sized optotypes that are “currently” displayed, and an optotype one size larger than the two larger displayed optotypes are evaluated. To meet rule four, two of the responses must be correct. If the largest optotype is the largest optotype on the eye chart, rule four is not met, even if two of the responses are correct. If rule four is met, the platform may be programmed to advance the user to the “breakout phase” as described below. If rule four is not met, the platform may evaluate whether the criteria for rule four are met.

In preferred embodiments, rule five governs the potential advancement to the “breakout phase” under specific performance patterns during the descending staircase phase. Rule five allows for “breakout” based on responses to larger optotype sizes, when downward progression has produced limited or ambiguous data. The platform initiates a rule five evaluation only if the most recent three optotype response outcomes conform to one of the following sequences: PFF (Pass-Fail-Fail), FFF (Fail-Fail-Fail), or FFP (Fail-Fail-Pass), as these sequences indicate declining or inconsistent visual performance across the smallest optotype sizes tested.

Additionally, to initiate a rule five evaluation, the two smaller optotype sizes previously displayed must have been presented only once each, constituting isolated data points at or near the bottom of the chart, thereby lacking sufficient presentation density for reliable threshold determination.

Upon satisfaction of the aforementioned rule, five conditions, the system evaluates the user's performance at the largest optotype size “currently” displayed, along with the two lines located one and two levels above that size. To satisfy Rule Five, the platform must confirm that a minimum of two optotype responses have been recorded at each of the three lines under evaluation, such that at least two of the six responses must be correct, also referred to as “passes”, indicating successful letter recognition.

If the aforementioned conditions for rule five are met, and the testing has not reached the topmost line of the acuity chart, the platform may evaluate responses using rule five to determine whether a user may progress from the descending phase to the breakout phase.

If rule five is not met, the descending phase continues by sequentially presenting smaller optotype lines until enough data is gathered to satisfy one of the breakout rules. In some embodiments, the system may generate additional letter presentations at the current or nearby lines (above or below) to ensure the minimum required number of responses (usually 2 per line at 3 consecutive levels) is achieved, thereby helping to trigger a rule once more data is collected. The process may continue in an iterative loop, adding data, reassessing against the rules until one of the breakout rules is satisfied, or the smallest line size available is reached.

In embodiments wherein the platform detects that any of the three lines under evaluation are clearly failing (i.e., 4+trials with 0 or 1 PASS), the system may bypass the standard rules and trigger breakout at the line above, i.e., one size above the size of the largest failing optotype. Thereby, providing a safety mechanism to avoid unnecessary additional testing when it is evident that the user cannot reliably identify letters at a given level.

In preferred embodiments, the platform will also be governed by rule six. Rule six, a safeguard rule, ensures that the system does not misinterpret three correct answers in a row (PPP) as a reason to advance into the breakout phase, unless a specific set of conditions is met. For example, although three correct responses in a row may indicate strong performance, rule six does not allow the system to continue to breakout based on the three passes unless the current testing is already at the bottom of the chart, or the previous 3 responses were all fails (FFF) or 1 pass followed by 2 fails (PFF). Such specific conditions prevent the system from determining a breakout algorithm based on luck or guessing.

Furthermore, if completing five total letter presentations per optotype during testing requires fewer or more than three letters to be displayed at a time, the system shall preferably present letters in batches of three to complete the exact number of required presentations at both the threshold line and the line immediately below. In some embodiments, near test completion, one or two letters may be presented with crowding bars, as described below. Regardless of rule satisfaction, if any of the three lines under evaluation are determined to be clearly failing (defined as four or more responses with zero or one correct), the system will trigger breakout from the line immediately above the failing line.

The following are exemplary embodiments of sample descending phase responses. Such exemplary embodiments are intended to clarify the herein described rules and platform algorithms and are not intended to limit the scope of the present disclosure. For the purposes of the following examples a Pass or “P” refers to the users responding correctly to a presented optotype, and a Fail of “f” refers to a user responding incorrectly to a presented optotype.

During the descending staircase phase, specific letter response patterns dictate the progression of optotype groupings, in preferred embodiments, optotypes will be presented in vertical rows composed of preferably one optotype per line. For example, when a response pattern of Pass, Pass, Pass (P,P,P) is observed—such as successful identification on lines 07, 06, and 05, such that one letter from line 07 is set on the top of the vertical column, one letter from line 06 was set in the center if the vertical column and one letter from line 05 was set on the bottom of the vertical column, the system may then proceed to the next smaller grouping, which in this exemplary embodiment would be lines 04, 03, and 02, also set in a vertical column composed of one optotype per row. But, in embodiments wherein the test has reached and passed lines 01, 00, and −01 once, the same group may be repeated once more for confirmation.

Furthermore, at the bottom of the chart, if only partial responses exist across these lines (i.e., two responses present for some but not all three lines), the system will preferably repeat the set. However, in embodiments wherein there is exactly one PASS on two lines above the bottom, and exactly two responses each on the bottom line and the line above it, the breakout may occur using those bottom two lines. Additional exceptions include continuation at the smallest optotype if failure occurred at or below that level, or breakout under certain multi-line fail/pass conditions that extend above and below the displayed grouping.

In some embodiments, in the case of a Fail, Fail, Fail (F,F,F) sequence (e.g., lines 07, 06, and 05), the system may shift upward, framing the last letter from the previous larger line grouping, typically resulting in the next presentation of lines 09, 08, and 07. If this occurs at the top of the chart (lines 10, 09, 08) and all three lines are failed, the group is repeated once more. In early testing (first or second responses), the system may jump to the largest available optotype, or a line size approximately double the previously displayed size. If an intermediate “top line” grouping contains a partial pass and no fails above it, and insufficient responses exist to proceed, the system may advance upward by only one line instead of two.

In some embodiments, when a response pattern of Pass, Pass, Fail (P,P,F) occurs—such as on lines 06, 05, and 04—the smallest passing line is framed (e.g., line 05), and the same three lines (06, 05, 04) are repeated. If a pattern of Pass, Fail, Pass (P,F,P) is recorded—such as 03, 02, 01—the same group is repeated with a fail-based framing. Similarly, for Pass, Fail, Fail (P,F,F), the highest pass (e.g., line 05) is framed and the system moves upward accordingly to lines 06, 05, and 04. Extended exceptions permit the repetition of lines when specific pass/fail response distributions exist within limited top line conditions.

In some embodiments, a Fail, Pass, Pass (F,P,P) pattern—such as 06, 05, 04—may suggest distraction or random guessing. In such cases, the smallest pass is framed, and the system proceeds with the next group as 04, 03, 02. Conversely, a Fail, Fail, Pass (F,F,P) pattern often implies a lucky guess or inconsistent attention. The system compensates by returning to a prior higher grouping (e.g., 08, 07, 06) to validate acuity and stabilize the logic path. If the largest display letter was the initial fail, that grouping is repeated for confirmation.

In some embodiments, in a Fail, Pass, Fail (F,P,F) pattern—such as 04, 03, 02—the largest fail (e.g., line 04) is retested, and the test proceeds upward accordingly to lines 05, 04, and 03. Special exceptions apply at the smallest optotype levels (e.g., 20/16). If line 20/16 must be presented as the first or second letter in a grouping during the descending phase, its pairing will vary based on position, in some embodiments the pairing 20/20 with 20/16, in other embodiments the pairing may be 20/16 with 20/20.

In embodiments where any single breakout rule is satisfied, as described above, the examination may enter the breakout phase, such that the system may present groups of three optotypes of identical size, vertically aligned with crowding bars, as described below. If fewer than or more than three letters are required to complete exactly five presentations at both the threshold line and the line immediately below, the platform displays as many as necessary—never exceeding three at once. Near test completion, one- or two-letter presentations are permissible.

As described above, after two presentations per line in the descending staircase, the platform determines the letter size for the breakout phase, according to the following priority-ordered breakout rules. In preferred embodiments, if the chart's smallest line, line −01, has been passed on two separate presentations, the breakout phase grouping begins exclusively with optotypes of that line size (−01, −01, −01). Similarly, if the largest line (line 10) has been failed twice, the initial breakout phase grouping preferably begins with the same largest line (10, 10, 10).

In embodiments wherein the smallest line (−01) is not passed twice, and the largest line (10) is not failed twice the breakout phase optotype phase will vary depending on which optotypes were passed and/or failed during the aforementioned descending phase. In embodiments where in the smallest presented line presented twice and passed twice, is passed twice: , the next three letters, presented for the breakout phase, may be displayed at that smallest size. For example, if lines 04 (P F), 03 (F P), and 02 (P P) were presented, the subsequent breakout phase grouping may be 02, 02, 02.

In embodiments wherein any line, corresponding to a particular visual acuity score, has accrued two fails, it is preferable that the platform be programmed to select the next larger line that possesses at least one PASS and presents three letters at that size for the initial breakout phase. For example, if line Lines 03, 02, 01 are presented, and line 01 is failed twice while line 02 has one pass; the next grouping displayed is preferable 02, 02, 02.

In embodiments wherein no line has two fails, yet one or more lines have two passes, the grouping for the breakout phase is preferable one line below the smallest line with two passes.

For example, if lines 04 (PP), 03 (P P), and 02 (F P) are observed, the system may preferably present three optotypes in a vertical orientation, as herein described, having a size of 02, 02, 02. Similarly, in an embodiment where line 03 holds only one PASS, the grouping would adjust to 03, 03, 03.

In embodiments wherein each of the three lines has exactly one pass and one fail, the system may preferably select the intermediate line (by size) for next three-letter grouping for the initial breakout phase presentation. For example, if a user has one pass and one fail at line 06, one fail and one pass at line 05, and one fail and one pass and line 04, the initial breakout phase grouping is preferably 05, 05, 05. In preferred embodiments, this rule equally applies at the chart's top or bottom, producing groupings 09, 09, 09 (top-line case) or 00, 00, 00 (bottom-line case) as appropriate.

In some embodiments, when testing has “descended” to the two bottommost lines of the chart and either line lacks two recorded responses, the system interprets any missing response as a fail for rule evaluation.

After the aforementioned initial breakout phase optotype sizes are presented and tested, the breakout phase continues presenting screens, preferably with three optotypes each, set in a vertical column. As described above, the optotype size of the initial breakout screen is based on the responses during the descending phase. In preferred embodiments, as described above, the platform may show three optotypes of the same size or differing size. In preferred embodiments, after each group of three letters, the system tracks how many were correctly identified (passes) vs. failed. Depending on the results, the platform may rely on the response to data to determine whether to maintain the current optotype size for the next presentation, display a smaller and therefore harder set of optotypes for the next presentation, or display larger, and therefore easier optotypes for the next presentation.

Once breakout begins, up to 3 letters of the same or different sizes will be shown vertically with crowding bars, depending on the correctness of previous responses.

The goal of the breakout phase is to get responses to five letters at the threshold line, the smallest size that a user can mostly read, meaning a line wherein users can read three out of the five optotypes, and five responses at the line size below the threshold line. In preferred embodiments, optotypes may be shown in groups of 3, or fewer if needed to make 5 total, but it is preferable that users will not be shown more than three optotypes at once, preventing inaccurate results due to eye fatigue, distraction, etc. It is preferable that a user does not see more than 3 letters at once.

Once threshold, defined as one or more missed optotypes within a given presentation, is reached. The protocol preferably assigns the final visual acuity result based on the smallest LogMAR line where at least 3 out of 5 optotypes are correctly identified, designating this as the threshold line. To confirm the threshold, the participant must fail at least 3 out of 5 optotypes on the line immediately below. Each visual stimulus and response are preferably documented by the software, which automatically resets to test the contralateral eye upon completion. Additionally, a timer function is integrated to measure the total “time to assess visual acuity” enabling precise tracking of test efficiency.

In preferred embodiments, if the user performs well, such as two or three passes, the system may descend to the next smaller line to test finer vision. But if performance is poor the system may ascend back to the line one size above the largest line currently being displayed to recheck. Once five letters have been tested at both the breakout line and the line below, the system preferably has enough data to calculate a visual acuity score, based on the line with the appropriate number of passes.

It is preferable that three optotypes be presented per screen for testing. If the larger of the two lines has two passes, and the smaller has two failed, breakout continues with the size of the larger line. In some embodiments, if the larger line has two passes, and the smaller line registers exactly one pass, breakout continues with the optotype size of the smaller line. If the smaller line alone achieves two passes, breakout phase size continues with the size of the smaller line. If the larger line holds fewer than two passes, and the smaller line has two fails, the breakout phase continues in descending mode starting from the line above the bottom two lines. If neither of the bottom two lines achieves two passes or two fails, the test likewise continues descending from the line above.

In preferred embodiments, the platform's standard logic protocol may preferably be designed to display three optotypes in descending size to facilitate rapidly identifying visual acuity threshold, and to prevent boredom and fatigue. Visual acuity testing for lines larger than 20/64 may not allow simultaneous display of 3 same sized optotypes, on many current smartphones which lengthens the protocol's testing time, and therefore to optimally utilize the smartphone testing protocol design, it is preferable to begin testing “in the middle” of the eye chart. Such embodiments are preferable, and the majority of users will have visual acuity levels closer to 20/20 than 20/200. Additionally, smaller visual acuity lines may display three letters simultaneously which may significantly reduce test time. Furthermore, displaying three optotypes a time allows greater accuracy and detection of small changes as such a display preferably facilitates best three out of five design to pass a particular acuity line, is consistent with ETDRS protocol, and allows true LogMar comparisons with accurate estimation of acuity “between lines”.

In some embodiments, the described platform may include a protocol for expedited screening, such that the platform may first display a single large optotype. It is preferable that the optotype be either twice the size of the test line or the maximum optotype size that may be displayed given screen size constraints, whichever is smaller. The large optotype is preferably displayed a maximum of two times. In embodiments wherein the user incorrectly identifies the large optotype twice, the user may be referred for professional examination. In embodiments wherein the user correctly identifies the first or second presentation of the large optotype, the platform may proceed to a modified “descend mode”, wherein the largest optotype presented is either selected by the user alternatively, automatically selected based on the user's age. In some embodiments, the platform may employ the aforementioned descending phase protocol (breaking out when appropriate) as defined, herein, without moving “up” in the chart, as is described above for the classic “descending staircase mode. For example, if a user “fails” the top line, the platform may report that the vision is “worse than 20/xx without testing higher visual acuity levels, and the assessment for that eye is concluded.

In some embodiments, a LogMAR value may be determined, as well as a computation of reliability as described herein. In some embodiments, such modified, expedited protocols may provide an exact visual acuity assessment if vision is equal to or better than the starting line size and can be performed in 20 to 40 seconds per eye.

In some embodiments, the described platform may additionally include a myopia progression monitoring protocol, such that the platform may be programmed to detect and measure accommodation and focusing differences by presenting optotypes consisting of various chromatic qualities. In some embodiments, to monitor myopia progression, the “breakout” portion of the platform may include three optotypes of differing colors simultaneously, such that the platform may quantify shifts in visual acuity for one or more colors and “translate” those shifts into myopia progression, direction, and severity.

For myopia monitoring, in some embodiments, the platform may first obtain an exact visual acuity score for each eye using red optotypes, followed by green optotypes, and then followed by white, or alternatively grey, optotypes. It is preferable that the aforementioned visual acuity testing be performed on a pure black background at a distance of 4 meters using the “Extended Protocol” as described herein. Measurements serve as a baseline, a start of treatment, or other monitoring process. As described herein, immediately following the 3 independent visual acuities tested for each eye, each eye may then independently be presented with three optotypes simultaneously, one red, one white (or grey), and one green in color against a pure black background. The starting size of each optotype is determined by either the baseline acuity for the specific color and eye, as described above, or by the last monitoring measurement obtained for the specific color and eye.

In some embodiments, the first sequence of the monitoring process consists of five randomized presentations of three optotypes of appropriate size and color. For example, in some embodiments, the first sequence may contain a red optotype on top, a white (or grey) optotype in the middle, and a green optotype at the bottom. It is preferable that in each subsequent presentation of optotypes, the top and bottom optotype colors reverse the “position” with the white optotype remaining in the middle.

After the first sequence, each “color” is analyzed and the next size for each color is either one-line larger if the patient incorrectly identified three or more optotypes, or one-line smaller if the patient correctly identified three or more optotypes. In preferred embodiments the aforementioned process continues for a maximum of two-lines from starting size for each color. In embodiments wherein one or two colors has “completed” testing after two sequences, a third sequence is presented, still containing all three colors at sizes not previously displayed for those colors . . . preferably larger or, alternatively, smaller. For those optotypes that have “completed” testing, this third sequence of information is not needed for scoring but is preferably included for proper testing procedure. At the completion of much myopia management testing, both visual acuity scoring and LogMar value may be calculated by the platform calculated as described herein.

As seen in FIG. 1, in preferred embodiments, each optotype, both in the descending phase and breakout phase of the herein described platform, may be surrounded by crowding bars, such that in preferred embodiments, each optotype 11, 12, and 13, may be flanked, on the top, bottom left, and right, by a line, such that the top of each optotype is set below a horizontal line, the bottom of each optotype is set above a horizontal line, and the sides of each optotypes are each set adjacent to vertical line.

In preferred embodiments, the system may display visual optotypes accompanied by crowding bars, wherein the stroke width of each crowding bar is equal to 20% of the optotype stroke width, maintaining consistent proportionality relative to the target symbol. The spacing between the edge of each optotype and its corresponding crowding bars is defined as a selectable percentage, preferably, depending on the embodiment, fifty percent, seventy five percent, or one hundred percent, representing the relative distance from the optotype to the surrounding crowding frame. In preferred embodiments, all optotype types—including but not limited to symbols, HOTV characters, and SLOAN letters, may utilize a crowding bar distance set at fifty percent to optimize visual presentation on standard screen dimensions. During protocol validation under the Midwestern University study, HOTV optotypes were tested at both fifty percent spacing, to match the Amblyopia Treatment Study (ATS) protocol (e-ATS chart), and at one hundred percent spacing, to match the Early Treatment Diabetic Retinopathy Study (ETDRS) standard (e-ETDRS chart).

In embodiments relying on the aforementioned fifty percent crowding bar size configuration, such as for use on personal smartphones and other small screens, it is preferable that the platform architecture retain the capability to switch to one hundred percent crowding spacing to accommodate deployment on larger display formats, such as tablet computers or full-size monitors utilized in clinical environments. It is preferred that for any given test session, the crowding bar configuration remains consistent across all displayed optotypes.

Furthermore, it is preferred that there be no optotype repetition within a given acuity size group. It is preferred that each size grouping, includes a maximum of five presented optotypes per eye, and employ unique symbols or letters without duplication. In some embodiments, for efficiency and consistency across test levels, the same set of optotypes used for high-resolution lines (e.g., 20/16) may be reused for lower-resolution lines (e.g., 20/160), with direct correspondence also existing between 20/20 and 20/200, and so forth.

In preferred embodiments, it is preferable that the visual acuity test platform be set to a vertical screen orientation during testing. In preferred embodiments, optotype positioning may be determined as a function of three primary factors: (1) the specified Snellen line size (e.g., 20/100, 20/32), (2) the testing distance (in feet), and (3) the designated crowding bar percentage. It is preferred that optotypes be centrally aligned both vertically and horizontally.

In some embodiments, to efficiently utilize screen space while preserving the crowding effect required for accurate acuity assessment, vertically adjacent optotypes may feature overlapping crowding bars during distance testing. However, during near vision testing, it is preferred that optotypes be fully separated, and that crowding bars do not overlap. In preferred embodiments, regardless of distance and the line being tested, if two adjacent optotypes differ in size, the crowding bar spacing between them defaults to the value associated with the larger optotype.

The physical size (in inches) of each optotype, excluding the crowding bars, is calculated using the formula:

$$Optotype Height ( in ) = 1.75 \times ( x / 100 ) \times ( y / 20 )$$

where x is the numerator of the Snellen fraction representing the optotype size (e.g., for 20/40, x=40), and y is the test distance in feet.

In preferred embodiments, to render optotype placement and crowding parameters across a digital interface, a series of computations are performed using device-specific screen resolution values (PPI) and current Snellen line parameters. In the following calculation, TopImageSize refers to the size of the top optotype 11, as seen in FIG. 1. MidImageSize refers to the size of middle optotype 12, as seen in FIG. 1. BotImageSize refers to the size of bottom image 13, as seen in FIG. 1. CB50 refers to a crowding bar size that is 50% of the optotype size. Similarly, CB100 refers to a crowding bar size that is 100% of the optotype size. In preferred embodiments, the positioning and sizing steps may include the following:
1. Compute TopImageSize, MidImageSize, and BotImageSize using the standard optotype height formula, converted to pixels via multiplication by PPI.
2. Derive TopCrowdBar, MidCrowdBar, and BotCrowdBar by multiplying the respective image sizes by 0.2 (20%).
3. For crowding configurations, compute both 50% and 100% image size groupings:
   50% crowding: Multiply each image size by 2.4
   100% crowding: Multiply each image size by 3.4
4. Determine total display height for both 50% and 100% crowding configurations by summing the adjusted image sizes and subtracting overlap areas defined by MidCrowdBar and BotCrowdBar.
5. Calculate top optotype vertical start position (TopOptoTop) by centering the total height within the screen.
6. Calculate horizontal centering (TopOptoLeft, MidOptoLeft, BotOptoLeft) by subtracting each respective optotype size from the screen width and dividing by two.
7. Compute vertical positioning of the middle and bottom optotypes by incrementing from the top position, subtracting the relevant crowding bars to preserve overlap logic.

In some embodiments, the specific computational steps may be outlined numerically as follows:

$$TopImageSize = round ( \{ [ ( x / 100 ) \times 1.75 \times ( distance in feet ) ] / 20 \} \times PPI , 0 )$$

$$TopCrowdBar = round ( TopImageSize \times 0.2 , 0 )$$

$$MidImageSize = [ same formula as above ]$$

$$MidCrowdBar = MidImageSize \times 0.2$$

$$BotImageSize = [ same formula ]$$

$$BotCrowdBar = BotImageSize \times 0.2$$

$$CB 50 TopImageSize = round ( TopImageSize \times 2.4 , 0 )$$

$$CB 100 TopImageSize = round ( TopImageSize \times 3.4 , 0 )$$

$$[ Repeat similar logic for mid and bottom ]$$

$$CB 50 TotalHeight = sum of CB 50 Top , Mid , and Bot image sizes - MidCrowdBar - BotCrowdBar$$

$$CB 100 TotalHeight = [ as above with 100 \% values ]$$

$$CB 50 TopOptoTop = int ( ( ScreenHeight - CB 50 TotalHeight ) / 2 )$$

$$CB 100 TopOptoTop = int ( ( ScreenHeight - CB 100 TotalHeight ) / 2 )$$

$$[ Continue as specified through CB 50 BotOptoLeft and CB 100 BotOptoLeft ]$$

Once the breakout phase is completed, as described above, it is preferable that the system may compute a quantitative visual-acuity value using the five-letter, line-by-line logarithmic method adapted from Holladay et al., J. Refractive Surgery. The visual acuity calculation is preferably based on the threshold line, the smallest optotype line on which the user accurately recognizes at least three of five letters during breakout testing. As known to those familiar with the line-by-line logarithmic method, each acuity “line” is pre-assigned a base LogMAR value (e.g., 20/20→0.00; 20/25→+0.10; 20/200→+1.00). The threshold line's LogMAR base value preferably constitutes the initial score.

Additionally, to refine the score and achieve letter-level resolution, the algorithm may preferably apply an additional fixed increment of 0.02 LogMAR per letter, such that letters missed on the threshold line add +0.02 LogMAR each to the final visual acuity score. Furthermore, correct letters on the line immediately smaller than threshold subtract 0.02 LogMAR each to the final visual acuity score. The five individual letter contributions are summed and divided by five to yield a single, final LogMAR score, suitable for statistical analysis and direct line-to-line comparison. In some embodiments, for clinician readability, the computed LogMAR value may be reconverted to the nearest Snellen or decimal equivalent; however, LogMAR remains the authoritative value for data storage and study statistics.

In preferred embodiments, as described above, the described platform may consider all responses recorded during the test, rather than relying solely on a line-by-line scoring method. To ensure reliability, all responses may be categorized as either, false positive (a correct response smaller than threshold), a true negative (an incorrect response smaller than threshold), a true positive (a correct response larger than threshold), or a false negative: an incorrect response larger than the threshold. The category of the responses, and the associated scoring method, as described below, may determine the test's reliability index. The reliability index is preferably a quantitative score used to assess how trustworthy or consistent a user's responses were during a visual acuity test. In preferred embodiments, the reliability index may help users determine whether the test results are valid or potentially unreliable due to inconsistent or implausible responses.

For purposes of response classifications, “smaller than threshold” is defined as any optotype displayed at a size that is less than the measured threshold in logMar minus 0.12 logMar, such that “smaller than threshold” is defined as 1.2 lines smaller than the measured threshold visual acuity. Additionally, “larger than threshold” is defined as any optotype displayed at a size that is more than measured threshold in logMar plus 0.14 logMar, such that “larger than threshold” is more than 1.4 lines larger than the measured threshold visual acuity. This 0.14 logMar “buffer” is preferred as it may consider optotypes that are difficult to recognize. Moreover, false responses are statistically weighted based upon their “distance” from measured threshold.

In preferred embodiments, all correct (true positive and true negative) responses that are greater than 0.14 and 0.12 logMar respectively from threshold are assigned a value of 1. Conversely, all incorrect (false negative and false positive) responses that are greater than 0.14 and 0.12 logMar respectively from threshold are assigned a value of 1 minus the distance from threshold (logMar). The closer to threshold, the value for that response is closer to 1. The further from threshold, the value for that response is further from 1. To ensure that the distance from threshold does not exceed 1 (resulting in a negative valuation), the distance from threshold is divided by the maximum possible distance (in logMar) prior to being subtracted from the initial value of 1. The result is the numerator of our reliability index.

To calculate the denominator or maximum possible (perfect) “score”, each false positive response is calculated as if it were a true positive (value of 1) and similarly, each false negative response is calculated as if it were a true negative (value of 1). These responses are added to one another to provide us with the denominator of our reliability index. By considering all responses defined as “smaller than threshold”, a “False Positive Reliability Index” may be calculated. Similarly, by considering all responses defined as “larger than threshold”, a “False Negative Reliability Index” may be calculated. And finally, by considering all responses as defined above, an “Overall Reliability Index” may be calculated.

In some embodiments, the herein-described platform may include protocols for testing near vision, contrast sensitivity, Vernier acuity, stereopsis, convergence, accommodative amplitude, color, focal length determination, binocularity, and other visual functions.

In some embodiments, the size of the optotypes may adapt in response to the distance between a user and the screen. In other embodiments, it may be required that a user stand a particular distance from the screen, exemplary distances may include one foot, two feet, ten feet, twenty feet etc. Distances may be determined by the user's needs, the screen size, and the starting line of the tested optotypes.

In some embodiments, the platform may be programmed with a timer, including but not limited to an internal timer, such that the timer records the time it takes each eye to correctly or incorrectly determine an optotype. In some embodiments, the timing may be factored into determining a user's final visual acuity score. In some embodiments, the platform may be individually calibrated to account for a user's screen size and testing distance. In preferred embodiments, the platform may be adaptable for use from forty centimeters to six meters.

The platform, as described herein, may function to perform an eye exam at a rate that is approximately forty percent faster than traditional eye exams currently known in the art, while maintaining the accuracy of the “gold standard” tests currently known in the art. Additionally, the herein described platform is preferable as the herein described platform provides greater inter-tester reliability and correlation than standard manual methods, such that the herein described platform is reliable even when administered by novice first time users, such as but not limited to parents and teachers not proficient in the art of eye exams.

In preferred embodiments, the platform described herein may analyze groupings of optotypes, such that the platform algorithm analyzes a user's responses to multiple lines to determine the best optotype size for continued presentation. Such embodiments are preferable, as they accelerate testing time, when compared to traditional testing methods, thereby avoiding patient fatigue, and providing more reliable results.

In preferred embodiments, the described platform may display up to 3 optotypes simultaneously, such that the optotypes are arranged in one (or more) vertical column(s), with each optotype being of a different size. In preferred embodiments, regardless of specific presentation, optotypes of differing sizes may be presented largest to smallest (top down). Such an orientation is preferable, as the aforementioned top to bottom orientation is congruent with a user's tendency to “read” from the top down. Furthermore, such an orientation is congruous with the nature of human vision, such that users naturally first look at objects that are larger and easier to see, before looking at objects that are smaller, and more challenging to see. Embodiments wherein optotypes of different size are presented simultaneously are also preferred, as such embodiments provide a psychometric design perspective, preventing test performance from being impacted by distractions, inattention, boredom, and/or user fatigue.

In some, preferred embodiments, the described method and algorithm may measure a user's response times to acquire data sets. Response times are known to take longer as the sensory stimulus approaches the sensory threshold, such that the algorithm may consider a user's response time to assess a user's visual performance. Additionally, response times for right eye, left eye and overall assessments may be made and tracked for changes over time. In some embodiments, such response times may be integrated into the reliability index. In some embodiments, the algorithm may be programmed to factor in slower response times and more false negative fluctuations in one eye, both eyes, or over time to indicate early vision conditions even if visual acuity remains acceptable or unchanged.

In some preferred embodiments, the described platform may perform a vision exam by rapidly alternating optotype presentation between a user's left and right eyes for controlled psychometric comparison. In such embodiments, rapidly alternating testing between the two eyes may standardize the testing environment to equalize testing conditions between the two eyes. In some embodiments, the screen displaying the optotypes may be virtual reality goggles, red/blue lenses, or polarized glasses, such that rapid alternating displays of one or more optotypes between eyes may be easily displayed. Such a method may be preferable to assess and track for visual acuity, contrast issues, and to track changes over time for Amblyopia (lazy eye).

1-44. (canceled)

45. A digital platform for determining a subject's visual acuity, said platform including;
a screen that presents groups of a series of grouped optotypes to a user;
said screen being programmed with a plurality of algorithms, whereby the plurality of algorithms are programmed to analyze the responses to the presented groups of optotypes to determine future optotype presentations for testing;
the plurality of algorithms including a first algorithm for a first series of grouped optotype presentation, wherein the first series comprise an adaptive threshold determining phase, herein referred to as a first descending phase of testing, and a second algorithm for a for a second series of grouped optotype presentation, wherein the second series comprises a second threshold refining phase, herein referred to as the breakout phase of testing; and
the plurality of algorithms further including an algorithm for calculating a user's visual performance.

46. The digital platform of claim 45, wherein the presented optotypes are presented in a vertical column, wherein one optotype is set per line of said column, and the vertical column is comprised of three optotypes set in a single vertical row.

47. The digital platform of claim 45, wherein the second algorithm calculates the optotype size for presentation during the second refining threshold phase, based on the user's responses to the optotypes presented during the first threshold-determining phase.

48. The digital platform of claim 45, wherein said digital platform is programmed to present optotypes in accordance with a LogMAR protocol for decreasing and increasing optotype size.

49. The digital platform of claim 45, wherein the digital platform presents optotypes of increasing and decreasing size, wherein the difference in size between optotypes of approximately 0.1 LogMAR.

50. The digital platform of claim 45, wherein the presented optotypes vary in color.

51. The digital platform of claim 50, wherein the presented optotypes, presented in a single vertical column vary in color.

52. The digital platform of claim 51, wherein the colored optotypes are presented on a colored screen.

53. The digital platform of claim 45, wherein the optotypes are positioned in a vertical column such that the largest optotype is positioned at the top of the column, the mid-sized optotype is positioned at the center of the column, and the smallest optotype is positioned on the bottom of the column, wherein largest optotype is 0.02 LogMAR larger than the mid-sized optotype, and the mid-sized optotype is 0.02 LogMAR larger than the smallest optotype.

54. A digital platform for assessing a user's visual performance, the platform comprising a display module configured to present a series of vertically arranged columns of optotypes, wherein each column comprises optotypes of varying sizes, and wherein the selection of each subsequent column is dynamically determined based on the user's response to the preceding column, whereby each display module comprises a vertical single column, wherein the display platform displays optotypes in a single vertical row, with each line of the column comprising a single optotype, whereby the vertically arranged optotypes vary in size relative to one another, with a size difference of approximately 0.1 LogMAR between optotypes.

55. The digital platform of claim 54, wherein the user's visual performance is calculated based on a user's responses to the displayed optotypes as well as by the time it takes a user to respond to the displayed optotypes.

56. A method for determining a user's visual performance the method comprising a digital platform wherein the platform displays a series of screens, wherein each screen presents a vertical column comprised of optotypes, such that in each subsequent screen optotypes are grouped and regrouped in vertical columns, wherein the platform first presents a series of screens with shifting optotype sizes to determine a user's general vision threshold, and wherein the platform second presents a series of screens with a narrower size range of optotypes to further refine the users visual performance score.

57. The method of claim 56, wherein the presentation and scoring are completed in a total timeframe of between two and twelve minutes for testing a user's right and left eye.

58. The method of claim 56, wherein the method is performed to test a user's near vision, contrast sensitivity, vernier acuity, stereopsis, convergence, accommodative amplitude, color, focal length determination, and/or binocularity.

59. The method of claim 56, wherein in the first series of screens the top, middle, and bottom optotypes represent three adjacent LogMAR vision lines.

60. The method of claim 56, whereby repeated testing via grouping and regrouping a subsequent set of three vertically oriented optotypes on a display is administered in the first series of screens, whereby a smallest of the optotypes is smaller than an optotype that was failed on a previous display; and identifying a LogMAR threshold of visual acuity, and further displaying at least one optotype more than 0.1 LogMAR removed from the LogMAR threshold of visual acuity.

61. The method of claim 60, whereby said steps of grouping and regrouping in the first series of screens is repeated until at least ten optotypes are displayed with five optotypes evaluated on the LogMAR threshold with at least three out of five correct and whereby at least five optotypes on the adjacent line immediately below the LogMAR threshold are failed with less than three out of five correct.

62. The method of claim 56, whereby the second series of screens involves further displaying three optotypes, one at a time above the LogMAR threshold in descending fashion after the LogMAR threshold is obtained.

63. The method of claim 56, further comprising the step of identifying incongruent data points where either an optotype larger than the LogMAR threshold failed or an optotype smaller than the LogMAR threshold was passed and weighing incongruent data points by assigning them a false positive or false negative value depending on the incongruent data points distance from the LogMAR threshold.

64. The method of claim 56, whereby said step of repeating is conducted by visually displaying three vertically arranged optotypes, whereby said step of repeating is conducted by visually displaying three vertically arranged optotypes with the largest of three optotypes set at top and the smallest at bottom.
